SEQUENCE LISTING (12 sequences)

SEQ ID NO: 1 - DNA, 1138 bp, Homo sapiens
  source 1..1138: mol_type = genomic DNA
gcctcgctcaggctgccgatcggctcgttctctctgcgttgggaccgcggagggcattcgctgtgtgactcagggcaagtgtgcgcacctctctgagcctccgcggccttctctagcaaaatggtggagccggtacccggctgtaaggcaagctgggatgcgcggagtagcgggtggaaagctggactggagttctcactcggccgctgggtgacttcggtgcactagggatagtaacagtaccacctcgtagggttgtagaaagaccaagtgagttaatatttgtaaagtgtttagaacagcgtctactgcatggaaagtagtatttatttgttagagagactccaggaggtcttgcccagggattgacttctcccttccctgtctccaccccccacattcccgtttgtgagccaagccttgcttttgcaccggccgggctctcttgcctggaaatgcatacccatctcaaacctgggctttaaaatcacctcctctgggacgccttcctctacttctcacagttcaggcctgttctgccccacacagcagaaaatgcccacgatgcaataccagagtctgcatgggaccatttcaacaacccttagaagctgactcagctgtctcctggccctgacttttctaagatgagaggagctggtaggaacaatttgggaaccctcaaaggctgagagtatcccacccacttcacagatgaagcaaactgaggcccagagaagggaaattacttgcccaagatcacccagcaagtaagaaacagagctggagatgagctcaggccagcacggaaccacgcaaactccttctgtaggctttgtaaaggattgatgagaaaagcagatggttaagagctctacaacttctgtgagtttctccacgttggtcaggctggcctcgaactcccaccctcaggtgatccacctgccttggcctcctaaagttctgggattacaggcatgagccactgcacccaacctgaataatctattagagtctttatctctaagaagagaaatacatctgaactaaagggtaaaaaatggcatgttttaattggtaagtctaaatgcaaaacgatgacaataaaattttaaaggactgctaaaaaaaaaaaaaa

SEQ ID NO: 2 - DNA, 890 bp, Homo sapiens
  source 1..890: mol_type = genomic DNA
gcctcgctcaggctgccgatcggctcgttctctctgcgttgggaccgcggagggcattcgctgtgtgactcagggcaagtgtgcgcacctctctgagcctccgcggccttctctagcaaaatggtggagccggtacccggctgtaaggcaagctgggatgcgcggagtagcgggtggaaagctggactggagttctcactcggccgctgggtgacttcggtgcactagggatagtaacagtaccacctcgtagggttgtagaaagaccaagtgagttaatatttgtaaagtgtttagaacagcgtctactgcatggaaagtagtatttatttgttagagagactccaggaggtcttgcccagggattgacttctcccttccctgtctccaccccccacattcccgtttgtgagccaagccttgcttttgcaccggccgggctctcttgcctggaaatgcatacccatctcaaacctgggctttaaaatcacctcctctgggacgccttcctctacttctcacagttcaggcctgttctgccccacacagcagaaaatgcccacgatgcaataccagagtctgcatgggaccatttcaacaacccttagaagctgactcagctgtctcctggccctgacttttctaagatgagaggagctggtaggaacaatttgggaaccctcaaaggctgagagtatcccacccacttcacagatgaagcaaactgaggcccagagaagggaaattacttgcccaagatcacccagcaagtaagaaacagagctggagatgagctcaggccagcacggaaccacgcaaactccttctgtaggctttgtaaaggattgatgagaaaagcagatggttaagagctctacaacttctgtgag

SEQ ID NO: 3 - protein, 492 aa, Mus musculus
MDPSSKKVTGRLMLAVGGAVLGSLQFGYNTGVINAPQKVIEEFYNQTWNHRYGEPIPSTTLTTLWSLSVAIFSVGGMIGSFSVGLFVNRFGRRNSMLMMNLLAFVAAVLMGFSKLGKSFEMLILGRFIIGVYCGLTTGFVPMYVGEVSPTALRGALGTLHQLGIVVGILIAQVFGLDSIMGNADLWPLLLSVIFIPALLQCILLPFCPESPRFLLINRNEENRAKSVLKKLRGTADVTRDLQEMKEEGRQMMREKKVTILELFRSPAYRQPILIAVVLQLSQQLSGINAVFYYSTSIFEKAGVQQPVYATIGSGIVNTAFTVVSLFVVERAGRRTLHLIGLAGMAGCAVLMTIALALLERLPWMSYLSIVAIFGFVAFFEVGPGPIPWFIVAELFSQGPRPAAIAVAGFSNWTSNFIVGMCFQYVEQLCGPYVFIIFTVLLVLFFIFTYFKVPETKGRTFDEIASGFRQGGASQSDKTPEELFHPLGADSQV

SEQ ID NO: 4 - protein, 492 aa, Homo sapiens
MEPSSKKLTGRLMLAVGGAVLGSLQFGYNTGVINAPQKVIEEFYNQTWVHRYGESILPTTLTTLWSLSVAIFSVGGMIGSFSVGLFVNRFGRRNSMLMMNLLAFVSAVLMGFSKLGKSFEMLILGRFIIGVYCGLTTGFVPMYVGEVSPTALRGALGTLHQLGIVVGILIAQVFGLDSIMGNKDLWPLLLSIIFIPALLQCIVLPFCPESPRFLLINRNEENRAKSVLKKLRGTADVTHDLQEMKEESRQMMREKKVTILELFRSPAYRQPILIAVVLQLSQQLSGINAVFYYSTSIFEKAGVQQPVYATIGSGIVNTAFTVVSLFVVERAGRRTLHLIGLAGMAGCAILMTIALALLEQLPWMSYLSIVAIFGFVAFFEVGPGPIPWFIVAELFSQGPRPAAIAVAGFSNWTSNFIVGMCFQYVEQLCGPYVFIIFTVLLVLFFIFTYFKVPETKGRTFDEIASGFRQGGASQSDKTPEELFHPLGADSQV

SEQ ID NO: 5 - DNA, 1037 bp, Homo sapiens
  source 1..1037: mol_type = genomic DNA
tttgcttctaggaagcagaagactgaggaaatgacttgggcgggtgcatcaatgcggccaaaaaagacacggacacgctcccctgggacctgagctggttcgcagtcttcccaaaggtgccaagcaagcgtcagttcccctcaggcgctccaggttcagtgccttgtgccgagggtctccggtgccttcctagacttctcgggacagtctgaaggggtcaggagcggcgggacagcgcgggaagagcaggcaaggggagacagccggactgcgcctcagtcctccgtgccaagaacaccgtcgcggaggcgcggccagcttcccttggatcggactttccgcccctagggccaggcggcggagcttcagccttgtcccttccccagtttcgggcggcccccagagctgagtaagccgggtggagggagtctgcaaggatttcctgagcgcgatgggcaggaggaggggcaagggcaagagggcgcggagcaaagaccctgaacctgccggggccgcgctcccgggcccgcgtcgccagcacctccccacgcgcgctcggccccgggccacccgccctcgtcggcccccgcccctctccgtagccgcagggaagcgagcctgggaggaagaagagggtaggtggggaggcggatgaggggtgggggaccccttgacgtcaccagaaggaggtgccggggtaggaagtgggctggggaaaggttataaatcgcccccgccctcggctgctcttcatcgaggtccgcgggaggctcggagcgcgccaggcggacactcctctcggctcctccccggcagcggcggcggctcggagcgggctccggggctcgggtgcagcggccagcgggcgcctggcggcgaggattacccggggaagtggttgtctcctggctggagccgcgagacgggcgctcagggcgcggggccggcggcggcgaacaagaggacggactctggcggccgggtcgttggccgcggggagcgcgggcaccgggcgagcaggccgcgtcgcgctcacc

SEQ ID NO: 6 - DNA, 1608 bp, Homo sapiens
  source 1..1608: mol_type = genomic DNA
agctcctcccagcctcaggcccaggaatgggaatctctgtgggtcacacatcagtagggaggtctttcccgatccttttctatgctactccaggagtcaaagcgtctcctgggacttttcagggcgcttcagaagagccctgggcctaaaccagctcaaccaagctgcagggacccagcctcctgagaaaagtgaatgtgagcccggtgcattcagaggagaatgaagccttcacccagaacacactctgggaagatgtcccaggcccagggggagggtttgtactaccagacctaagtcacctaaactgacaccaagtctcatccatcccaaccattccattccgggtcagaggggtcatcgatttaaccagcaaggctgcccatccaacggttgctccctctgctccctggaagggcctcctcgtgggcgttctgtacctacaggtcttgttccgttctgggaactgccagtggtggcaagaggtggagcaacgggtgccagggcagggagaggtgagtctgggagggaagcagaggcaagatccatggggctttagagactttgccaaagcagtgcgactgctcccaggttgttgtcagccgtcaagagtgagtgcacctccctgggcagacttctgctgccccagtgcccaggaataggcaggggtttgccgcaaaatgaatgacacctggcagacaataagctgaagctttcattagcagcttaagctgaggactatctatgcaaccgatactccctgtgtgctccccgggactgcttaatgtgagcccttgtggagcgattggcaccaagaaagcaaggactaagtcagaagttcaagtcccagccttgccacagcctcagggtgccctcgagcacagcaagcctcagttttcccatctgtacaatgagagaggtacacaaggtagactcgaaggctctttgttgccagggccctgtgttcctttgagtgtatgtgcttctcaggcccacagaggtcctttgtgtttcgtatgtgaactgctctctaggaaacccatgtaactgtctgtgtcctggggcacatacatgaggactcatgtgggccgtattgtgtgtttgtgccggggggaggggagaccccagaacaatgtcccccaccccacccccctcctcaataggcggaagccactggcttcctccctttcctgcctcctgcctcctttgtgccagcaagactgagtactggagagagacaggggatgggaaaaatcagtccagctgtccccaggtctgcccttaccataaccttccccccacctcaagtgactcctcccaggccacacccatccccagccttgtgggggccagattggggggcctagaggctcaaaggcagaatgagtcctcccaccccctaccctgccacccctcccacccaagccacctcatttcctcttcctccccagcaccgacccacactgaccaacacaggctgagcagtcaggcccacagcatctgaccccaggcccagctcgtcctggctggcctgggtcggcctctggagtatggtctggcgggtgccccctttcttgctccccatcctcttcttggcttctcatgtgg

SEQ ID NO: 7 - DNA, 2510 bp, Homo sapiens
  source 1..2510: mol_type = genomic DNA
ctagtagcagaaacaaggtcctctggaagagcaactgatgctcttaggtactgaagcatcatcctgccccagagaccactcgcatatgaagcacacatattcagtctgccttacttgtgttaatgattgccagtgtccctctgacctcctagccctgaaaagtgtggcctgaaggtcatttcagagacggggagagctgctcagagaagccaatcggcgagtctaggacacacagacaggatctagtcccagagttcgctagcctaggtgagcgtcccctggccccttataccacttccttctccagcttgcatctaatctgctctggcagaccatcgtgtttcctgtcttcctggcagcctccagcacgctcagtgctactccctgcgcatgcgccctcctcccagtaccttctctgactccagtgggcttggagtgcgaggaggaagggtgaggaaggggtgaaatcaggtattggatccacagggggtctgaagagcactagcctggccttttgggactgaacttctgctatgaagacctccactgccatccctggagtccggggcacatccaaggcttgctgtccatcgtttactgtttacagatgacaacaatgactgtgttcggggcagaaatatccaccagggctagagtacaaaaggagtttgcattgatggccggacaggccctgtccctggcagcctgccagcgctgagtatgagacccagcgggaagtgctaccctggcagacgtgtccactgagtacacagaccaccaaggcaggcagctctcggggaagctgtctatgctgggccagcccaccttgagggcagggaacagaacagattgtggcagagaggaaaatgtggagcttctgtttgttcacagacacacgcactcgcccacgcacgcacgcacgcacgcacgcacgcacgaatgcacgcacgcagtagttgaatgctatggattccgctcagagctgagaacagccccagcgacagttccctggcctctctccttactctgatgtcctcatctgtcttcacatggtctcaggacgctaatactccatcctaatgtacactcctttccctgggcctccgttccagttcagttctcagaggacctggagggagtgattggctacaccaactttgctttcgttcaccaagcccatgtctctacttgggtgtctaatgggcatctccaacattacctaccccaaacagaaaaccctttcttccccccaaccacaccccaccctacccccacagtattttctccatgcccggaaagatctgctctcttatggtccctctttgcctcactgaaaagcaggacaagttggggacttcccaaacttttatgcatgaagaaacccaggcaatttgccaaaaggtacactctgggggtctgtcatttactctgagccagaaccctgaaatttttactaacccatcacataatgaatgaagagaatctttttctttttttttttttttctttttttttggtttttcgagacagggtttctctgtatagccctggctatcctggaacacactctgtagaccaggctggcctcgaactcagaaatccacctgcctctgcctcccgagtgctgggattaaaggcgtgcgccaccacgcctggctgaatgaagagaatcttgacctcatctccccagcctcttggtcctgagggaccctggtctacctactgctttgctgtcttcttagctcttcttacttttttgctgactcagacctatggctatctccattatacagatgaggagactgaggcatggatccctggttggtccatggtcacgtgaagcccatcacccagtatttgtaaagtgagatgggccaggctggtaccttggaactgaaactcacactgccctacctggaagaatctgacaggcaaaatctgctgctgaaagtgattgtctgtcacgtttctcagctgcccgactctgagaactccacagccccctttcgttccaccatactacagagtcgccacggaaagccggctctgtggagaagctgaggtagctgggtttctgtctgggttactctgtccagcgaggaaacaagtaccttagacccactaagcctctgctttctgaactgtaaagtgggggatatgacacctgcctcccagggatggctgaatgctctggcagaagcttagagcccccacagctacccctaggctcacagctcctccgatgagacctagaattgaggtatgagttgaataccccaggcaggtccaaggcttccacgggcccaggctgaccaagctgaggccgcccaccgtagggcttgcctatctgcaggcagctcacaaaggaacaataacaggaaaccatcccgaggggaagtgggccagggccagttggaaaacctgcctccctcccagcctgggtgtggctcccctctcccctcctgaggcaatcaactgtgctctccacaaagctcggccctggacagact

SEQ ID NO: 8 - DNA, 1479 bp, Homo sapiens
  source 1..1479: mol_type = genomic DNA
atggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtga

SEQ ID NO: 9 - DNA, 1124 bp, Homo sapiens
  source 1..1124: mol_type = other DNA
gcctcgctcaggctgccgatcggctcgttctctctgcgttgggaccgcggagggcattcgctgtgtgactcagggcaagtgtgcgcacctctctgagcctccgcggccttctctagcaaaatggtggagccggtacccggctgtaaggcaagctgggatgcgcggagtagcgggtggaaagctggactggagttctcactcggccgctgggtgacttcggtgcactagggatagtaacagtaccacctcgtagggttgtagaaagaccaagtgagttaatatttgtaaagtgtttagaacagcgtctactgcatggaaagtagtatttatttgttagagagactccaggaggtcttgcccagggattgacttctcccttccctgtctccaccccccacattcccgtttgtgagccaagccttgcttttgcaccggccgggctctcttgcctggaaatgcatacccatctcaaacctgggctttaaaatcacctcctctgggacgccttcctctacttctcacagttcaggcctgttctgccccacacagcagaaaatgcccacgatgcaataccagagtctgcatgggaccatttcaacaacccttagaagctgactcagctgtctcctggccctgacttttctaagatgagaggagctggtaggaacaatttgggaaccctcaaaggctgagagtatcccacccacttcacagatgaagcaaactgaggcccagagaagggaaattacttgcccaagatcacccagcaagtaagaaacagagctggagatgagctcaggccagcacggaaccacgcaaactccttctgtaggctttgtaaaggattgatgagaaaagcagatggttaagagctctacaacttctgtgagtttctccacgttggtcaggctggcctcgaactcccaccctcaggtgatccacctgccttggcctcctaaagttctgggattacaggcatgagccactgcacccaacctgaataatctattagagtctttatctctaagaagagaaatacatctgaactaaagggtaaaaaatggcatgttttaattggtaagtctaaatgcaaaacgatgacaataaaattttaaaggactgct

SEQ ID NO: 10 - DNA, 1703 bp, Homo sapiens
  source 1..1703: mol_type = genomic DNA
gtaaggcactggtctagaggaaataaggtgaagtcagatgatagcaataccactaccagtgcagtggcacatgcctgcactcccagctactacggaggctaaggtgggaggatcacttgagcccaggagtttgagaccagcatttatttatttactaacagactaaataaataaaccaacaataccaccaaccagtaaatgagaacttccatgctaaacacttcatgagcttatctggaaattgcacctctcctgatagcaatgagattggtgtgggaattccatttactcataagaagacagcctcggccgggctcagtggctcacgcctgtaatcccagtactttgggaggcgaggtgggtggatcacgaggtcaagagttcaagaccagcctggccaaaatggtgaaaccccgtctctactaaaaataaaaataattagccaggggtgatagcgggcgcctgtaatcccagctactcgggaggctgaggcagagaactgcttgaatccgggaggcggaggttacagtgagccgagatcgtgccgctgcactccagcctgggcgacagagcgagactctgtctgagaaaaaaaaaaattaaagaaaaaaacgaaaacagcctcactggccaggaactcgcccaggatcgcggccgggccagtatacagttgagctggttcaaacccgaggtctatcatgtccttaatgtcctatcatgtcagtcagttagcctgcctttcatattttccacacttacaggtcaccatagactcacctgaactgtgagaagtagaggaaggcgtcccagaggaggtgattttaaagcccaggtttgagatgggtatgcatttccaggcaagagagcccggccggtgcaaaagcaaggcttggctcacaaacgggaatgtggggggtggagacagggaagggagaagtcaatccctgggcaagacctcctggagtctctctaacaaataaatactactttccatgcagtagacgctgttctaaacactttacaaatattaactcacttggtctttctacaaccctacgaggtggtactgttactatccctagtgcaccgaagtcacccagcggccgagtgagaactccagtccagctttccacccgctactccgcgcatcccagcttgccttacagccgggtaccggctccaccattttgctagagaaggccgcggaggctcagagaggtgcgcacacttgccctgagtcacacagcgaatgccctccgcggtcccaacgcagagagaacgagccgatcggcagcctgagcgaggcagtggttagggggggccccggccccggcccctcccctcaccccctccccgcagagcgccgcccaggacaggctgggccccaggccccgccccgaggtcctgcccacacacccctgacacaccggcgtcgccagccaatggccggggtcctataaacgctacggtccgcgcgctctctggcaagaggcaagaggtagcaacagcgagcgtgccggtcgctagtcgcgggtccccgagtgagcacgccagggagcaggagaccaaacgacgggggtcggagtcagagtcgcagtgggagtccccggaccggagcacgagcctgagcgggagagcgccgctcgcacgcccgtcgccacccgcgtacccggcgcagccagagccaccagcgcagcgctgcc

SEQ ID NO: 11 - DNA, 3182 bp, Homo sapiens
  source 1..3182: mol_type = other DNA
gtaaggcactggtctagaggaaataaggtgaagtcagatgatagcaataccactaccagtgcagtggcacatgcctgcactcccagctactacggaggctaaggtgggaggatcacttgagcccaggagtttgagaccagcatttatttatttactaacagactaaataaataaaccaacaataccaccaaccagtaaatgagaacttccatgctaaacacttcatgagcttatctggaaattgcacctctcctgatagcaatgagattggtgtgggaattccatttactcataagaagacagcctcggccgggctcagtggctcacgcctgtaatcccagtactttgggaggcgaggtgggtggatcacgaggtcaagagttcaagaccagcctggccaaaatggtgaaaccccgtctctactaaaaataaaaataattagccaggggtgatagcgggcgcctgtaatcccagctactcgggaggctgaggcagagaactgcttgaatccgggaggcggaggttacagtgagccgagatcgtgccgctgcactccagcctgggcgacagagcgagactctgtctgagaaaaaaaaaaattaaagaaaaaaacgaaaacagcctcactggccaggaactcgcccaggatcgcggccgggccagtatacagttgagctggttcaaacccgaggtctatcatgtccttaatgtcctatcatgtcagtcagttagcctgcctttcatattttccacacttacaggtcaccatagactcacctgaactgtgagaagtagaggaaggcgtcccagaggaggtgattttaaagcccaggtttgagatgggtatgcatttccaggcaagagagcccggccggtgcaaaagcaaggcttggctcacaaacgggaatgtggggggtggagacagggaagggagaagtcaatccctgggcaagacctcctggagtctctctaacaaataaatactactttccatgcagtagacgctgttctaaacactttacaaatattaactcacttggtctttctacaaccctacgaggtggtactgttactatccctagtgcaccgaagtcacccagcggccgagtgagaactccagtccagctttccacccgctactccgcgcatcccagcttgccttacagccgggtaccggctccaccattttgctagagaaggccgcggaggctcagagaggtgcgcacacttgccctgagtcacacagcgaatgccctccgcggtcccaacgcagagagaacgagccgatcggcagcctgagcgaggcagtggttagggggggccccggccccggcccctcccctcaccccctccccgcagagcgccgcccaggacaggctgggccccaggccccgccccgaggtcctgcccacacacccctgacacaccggcgtcgccagccaatggccggggtcctataaacgctacggtccgcgcgctctctggcaagaggcaagaggtagcaacagcgagcgtgccggtcgctagtcgcgggtccccgagtgagcacgccagggagcaggagaccaaacgacgggggtcggagtcagagtcgcagtgggagtccccggaccggagcacgagcctgagcgggagagcgccgctcgcacgcccgtcgccacccgcgtacccggcgcagccagagccaccagcgcagcgctgccatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtga

SEQ ID NO: 12 - DNA, 2827 bp, Homo sapiens
  source 1..2827: mol_type = other DNA
gtaaggcactggtctagaggaaataaggtgaagtcagatgatagcaataccactaccagtgcagtggcacatgcctgcactcccagctactacggaggctaaggtgggaggatcacttgagcccaggagtttgagaccagcatttatttatttactaacagactaaataaataaaccaacaataccaccaaccagtaaatgagaacttccatgctaaacacttcatgagcttatctggaaattgcacctctcctgatagcaatgagattggtgtgggaattccatttactcataagaagacagcctcggccgggctcagtggctcacgcctgtaatcccagtactttgggaggcgaggtgggtggatcacgaggtcaagagttcaagaccagcctggccaaaatggtgaaaccccgtctctactaaaaataaaaataattagccaggggtgatagcgggcgcctgtaatcccagctactcgggaggctgaggcagagaactgcttgaatccgggaggcggaggttacagtgagccgagatcgtgccgctgcactccagcctgggcgacagagcgagactctgtctgagaaaaaaaaaaattaaagaaaaaaacgaaaacagcctcactggccaggaactcgcccaggatcgcggccgggccagtatacagttgagctggttcaaacccgaggtctatcatgtccttaatgtcctatcatgtcagtcagttagcctgcctttcatattttccacacttacaggtcaccatagactcacctgaactgtgagaagtagaggaaggcgtcccagaggaggtgattttaaagcccaggtttgagatgggtatgcatttccaggcaagagagcccggccggtgcaaaagcaaggcttggctcacaaacgggaatgtggggggtggagacagggaagggagaagtcaatccctgggcaagacctcctggagtctctctaacaaataaatactactttccatgcagtagacgctgttctaaacactttacaaatattaactcacttggtctttctacaaccctacgaggtggtactgttactatccctagtgcaccgaagtcacccagcggccgagtgagaactccagtccagctttccacccgctactccgcgcatcccagcttgccttacagccgggtaccggctccaccattttgctagagaaggccgcggaggctcagagaggtgcgcacacttgccctgagtcacacagcgaatgccctccgcggtcccaacgcagagagaacgagccgatcggcagcctgagcgaggcagtggttagggggggccccggccccggcccctcccctcaccccctccccgcagagcgccgcccaggacaggctgggccccaggccccgccccgaggtcctgcccacacacccctgacacaccggcgtcgccagccaatggccggggtcctataaacgctacggtccgcgcgctctctggcaagaggcaagaggtagcaacagcgagcgtgccggtcgctagtcgcgggtccccgagtgagcacgccagggagcaggagaccaaacgacgggggtcggagtcagagtcgcagtgggagtccccggaccggagcacgagcctgagcgggagagcgccgctcgcacgcccgtcgccacccgcgtacccggcgcagccagagccaccagcgcagcgctgccgcctcgctcaggctgccgatcggctcgttctctctgcgttgggaccgcggagggcattcgctgtgtgactcagggcaagtgtgcgcacctctctgagcctccgcggccttctctagcaaaatggtggagccggtacccggctgtaaggcaagctgggatgcgcggagtagcgggtggaaagctggactggagttctcactcggccgctgggtgacttcggtgcactagggatagtaacagtaccacctcgtagggttgtagaaagaccaagtgagttaatatttgtaaagtgtttagaacagcgtctactgcatggaaagtagtatttatttgttagagagactccaggaggtcttgcccagggattgacttctcccttccctgtctccaccccccacattcccgtttgtgagccaagccttgcttttgcaccggccgggctctcttgcctggaaatgcatacccatctcaaacctgggctttaaaatcacctcctctgggacgccttcctctacttctcacagttcaggcctgttctgccccacacagcagaaaatgcccacgatgcaataccagagtctgcatgggaccatttcaacaacccttagaagctgactcagctgtctcctggccctgacttttctaagatgagaggagctggtaggaacaatttgggaaccctcaaaggctgagagtatcccacccacttcacagatgaagcaaactgaggcccagagaagggaaattacttgcccaagatcacccagcaagtaagaaacagagctggagatgagctcaggccagcacggaaccacgcaaactccttctgtaggctttgtaaaggattgatgagaaaagcagatggttaagagctctacaacttctgtgagtttctccacgttggtcaggctggcctcgaactcccaccctcaggtgatccacctgccttggcctcctaaagttctgggattacaggcatgagccactgcacccaacctgaataatctattagagtctttatctctaagaagagaaatacatctgaactaaagggtaaaaaatggcatgttttaattggtaagtctaaatgcaaaacgatgacaataaaattttaaaggactgct

(19) United States
(12) Patent Application Publication
(10) Pub. No.: US 2026/0345820 A1
(43) Pub. Date: Nov. 5, 2026

(54) COMPOSITIONS AND METHODS FOR THERAPEUTIC VECTORS TARGETING BRAIN MICROVASCULATURE

(71) Applicant: THE TRUSTEES OF COLUMBIA UNIVERSITY IN THE CITY OF NEW YORK, New York, NY (US)

(72) Inventors: Umrao R. MONANI, Oradell, NJ (US); Shashi KUMAR, New York, NY (US)

(21) Appl. No.: 19/713,621

(22) Filed: Jun. 18, 2026

Related U.S. Application Data
(63) Continuation of application No. PCT/US2024/060834, filed on Dec. 18, 2024.
(60) Provisional application No. 63/612,264, filed on Dec. 19, 2023.
(60) Provisional application No. 63/612,277, filed on Dec. 19, 2023.

(51) Int. Cl. A61K 48/00; A61K 38/17; A61P 25/00; C12N 15/86

(52) U.S. Cl. CPC A61K 48/0033; A61K 38/1709; A61K 48/005; A61K 48/0075; A61P 25/00; C12N 15/86; C12N 2750/14143

(57) ABSTRACT

The present disclosure provides, inter alia, compositions and methods for improved therapeutic vectors for targeting brain microvasculature and employing such vectors to treat brain diseases.

### CROSS REFERENCE TO RELATED APPLICATIONS

The present application is a continuation of PCT international application no. PCT/US2024/060834, filed on Dec. 18, 2024, which claims benefit of U.S. Provisional Patent Application No. 63/612,264, filed Dec. 19, 2023, and U.S. Provisional Patent Application No. 63/612,277, filed Dec. 19, 2023. The entire contents of the above applications are incorporated by reference as if recited in full herein.

### FIELD OF INVENTION

The present invention is related to use of adeno-associated viral serotypes to target brain microvasculature.

### INCORPORATION BY REFERENCE OF SEQUENCE LISTING

This application contains references to amino acids and/or nucleic acid sequences that have been filed concurrently herewith as sequence listing XML file “CU24146-PCT-seq.xml”, file size of 29,840 bytes, created on Dec. 17, 2024. The aforementioned sequence listing is hereby incorporated by reference in its entirety pursuant to 37 C.F.R. § 1.52(e)(5).

### BACKGROUND OF THE INVENTION

The endothelial cells of the human brain microvasculature constitute an important component of the blood-brain barrier (BBB). This barrier restricts the entry of most circulating molecules into the brain parenchyma thereby ensuring the integrity and proper functioning of the cerebral circuitry. However, its existence also makes delivering therapeutic molecules to the brain parenchyma challenging. Defects of the brain endothelial cells compromise the BBB, often resulting in profound brain dysfunction, e.g., in Alzheimer's disease. Such defects can also cause disease without disrupting the BBB, e.g., in Glucose Transporter-1 deficiency syndrome (Glut1 DS). Efficiently and selectively targeting brain endothelia with therapeutic molecules is therefore an important aspect of treating neurovascular diseases. Brain endothelial cells can also serve as “factories” to express secreted proteins required for the health and viability of cerebral neurons. This is expected to be especially useful in treating lysosomal storage diseases (Gaucher's, Hurler's, Hunter's and Fabry diseases are examples), which affect brain neurons and stem from deficiencies of secreted enzymes.

Considerable effort has been expended in identifying viral vectors that efficiently target the mammalian brain microvasculature. Most studies have focused on adeno-associated viral (AAV) vectors given their approval for the treatment of neurodegenerative diseases such as spinal muscular atrophy (Zolgensma), muscle diseases such as Duchenne muscular dystrophy (Elevidys) and blood conditions, e.g., hemophilia (Roctavian, Hemgenix). While these efforts have yielded AAV serotypes capable of targeting brain endothelial cells, the overall outcome has disappointed; selectivity and efficiency were only assessed in restricted strains of the adult rodent, and the identified AAVs did not perform similarly in other species including primates. Failure of the AAVs previously reported to be brain endothelial—tropic is, at least in part, a consequence of the manner in which they were generated—by employing a screening process using a particular rodent strain or cultured cells derived from the rodent strain. Such engineered AAVs repeatedly failed to exhibit the tropism for brain endothelia beyond the cells/strains employed in the screen.

Thus, there exists a need for improved therapeutic vectors for targeting brain microvasculature and employing such vectors to treat brain diseases. This disclosure addresses these and other needs.

### SUMMARY OF THE INVENTION

Targeting brain endothelial cells in gene replacement type therapeutic strategies to produce and secrete proteins deficient in these diseases is appealing and clinically relevant. As disclosed herein, two naturally occurring adeno-associated viral serotypes, AAV5 and AAVrh74, are provided, which robustly and selectively target the mammalian brain microvasculature but not neuropil. These vectors are useful in the treatment of neurovascular and neurodegenerative conditions.

According to some aspects, the present disclosure provides AAV vectors with the potential to efficiently target brain endothelia broadly, across species and especially for the treatment of human brain diseases. In some embodiments, these AAV vectors were screened from a half dozen naturally occurring serotypes in neonatal and adult rodents of two distinct strains. As disclosed herein, two that robustly transduce brain endothelia when delivered systemically, without significant targeting of the brain neuropil (astrocytes and neurons), are AAV5 and AAVrh74, which are of considerable utility in delivering therapeutic molecules in a targeted manner to the human brain microvasculature for the treatment of common neurological conditions, e.g., Glut1 DS and Alzheimer's disease.

As disclosed herein, in some embodiments, AAV capsids are useful in the treatment of neurovascular or neurodegenerative diseases for which expression of therapeutic molecules in the brain microvasculature mitigates disease phenotypes. In some embodiments, AAV5 and AAVrh74 capsids target the primate and human brain microvasculature as shown here for the rodent. The embodiments disclosed herein are also useful for delivering therapeutic molecules for the treatment of lysosomal storage disorders, Glut1 deficiency and Alzheimer's diseases.

According to some aspects, the present disclosure provides a pharmaceutical composition comprising a recombinant adeno-associated virus (rAAV) vector carrying an agent that is effective to treat a brain endothelial dysfunction.

According to some aspects, the present disclosure provides a method of treating or ameliorating the effect of a brain endothelial dysfunction in a subject, comprising administering to the subject an effective amount of any of the pharmaceutical compositions disclosed herein.

According to some aspects, the present disclosure provides a pharmaceutical composition comprising a recombinant adeno-associated virus serotype 5 (AAV5) vector carrying an isolated nucleic acid comprising SLC2A1-NAT having a sequence selected from SEQ ID NO: 1 or SEQ ID NO: 2, or variants or fragments thereof.

According to some aspects, the present disclosure provides a pharmaceutical composition comprising a recombinant adeno-associated virus serotype rh74 (AAVrh74) vector carrying an isolated nucleic acid comprising SLC2A1-NAT having a sequence selected from SEQ ID NO: 1 or SEQ ID NO: 2, or variants or fragments thereof.

According to some aspects, the present disclosure provides a pharmaceutical composition comprising a recombinant adeno-associated virus serotype 5 (AAV5) vector carrying an isolated nucleic acid sequence encoding Glut1 operatively linked to a promoter.

According to some aspects, the present disclosure provides a pharmaceutical composition comprising a recombinant adeno-associated virus serotype rh74 (AAVrh74) vector carrying an isolated nucleic acid sequence encoding Glut1 operatively linked to a promoter.

According to some aspects, the present disclosure provides a method of treating or ameliorating the effect of Glucose transporter 1 deficiency syndrome (GLUT1DS) in a subject, comprising administering to the subject an effective amount of any of the pharmaceutical composition disclosed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows representative cortical sections from PND2 C57BL/6 mice injected (systemically) with either AAV1-eGFP, AAV5-eGFP or AAV9-eGFP. Whereas AAV9 transduces significant numbers of neurons (arrow) and glia (asterisk), AAV5 is more selective, transducing mainly the brain microvasculature. Depicted in the sections are brain capillaries stained for Glut1 and counterstained for GFP expression. Dose—1×10¹¹ GC.

FIG. 2 shows representative cortical sections from PND2 129SvTac mice injected (systemically) with either AAV1-eGFP, AAV5-eGFP or AAV9-eGFP. Whereas AAV9 transduces significant numbers of neurons (arrows) and glia, AAV5 is more selective, transducing mainly the brain microvasculature—even in this mouse strain. Depicted are brain capillaries stained for Glut1 and counterstained for GFP. Dose—1×10¹¹ GC.

FIG. 3 shows representative cortical sections from adult (8 wk) 129SvTac and C57BL6 mice injected (systemically) with either AAV5-eGFP or AAV-BI30-eGFP. AAV5 robustly and selectively transduces brain capillaries in both strains of mice. AAV-BI30, an engineered serotype efficiently transduces brain endothelia of adult C57BL/6 mice. Note: Depicted are brain capillaries stained for Glut1 or lectin and counterstained for GFP. Dose—1×10¹¹ GC.

FIG. 4 shows efficiency of transduction of the brain microvasculature by three naturally occurring AAV capsids. AAV5 was found to be significantly better than AAV9 at transducing the brain microvasculature in most of the comparisons done and tended to be more efficient than AAV1 as well. Note: n≥3 animals of each cohort. *, **, P<0.05, P<0.01 one-way ANOVA. Also note: Quantification was done on cortical sections by estimating the percent overlap between Glut1 signal (representing brain endothelia) and GFP label.

FIGS. 5A and 5B show representative brain sections from PND2 129SvTac mice injected (systemically) with either AAV-BI30-eGFP or AAVrh74-eGFP. FIG. 5A shows AAVrh74 was found to be significantly better than AAV-BI30 at transducing the brain microvasculature. Unlike AAVrh74, The BI30 serotype targeted significant numbers of neurons (arrows). Thalamic sections depicted for AAVrh74; cortical sections shown for AAV-BI30. FIG. 5B shows quantified comparison of efficiency with which each serotype transduced brain endothelia. Note: n≥15 fields from N≥3 mice of each cohort. *, P<0.05, t test.

FIG. 6 shows a 1.7 kb human Glut1 promoter element for use in Glut1DS and related therapies drives robust reporter expression. Note: **, ***, P<0.01 and P<0.001, one-way ANOVA, n=3 replicates for each experiment.

FIG. 7 shows the sequence (SEQ ID NO: 11) (entire) of the 1.7 kb human Glut1 promoter (SEQ ID NO: 10) (underlined) fused to the human Glut1 gene (SEQ ID NO: 8) (the rest). The construct is expected to raise Glut1 for the treatment of Glut1DS and other conditions characterized by low Glut1.

FIG. 8 shows the sequence (SEQ ID NO: 12) (entire) of the 1.7 kb human Glut1 promoter (SEQ ID NO: 10) (underlined) fused to the human SLC2A1-DT natural antisense transcript (SEQ ID NO: 9) (the rest). The construct is expected to raise Glut1 for the treatment of Glut1DS and other conditions characterized by low Glut1.

FIGS. 9A and 9B show a 1.7 kb human Glut1 promoter element drives robust expression of a reporter in vivo in brain endothelial cells. FIG. 9A shows representative photomicrographs of mouse brain cortical sections from animals injected with a 1.7 kb human Glut1 promoter driving eGFP in an AAV5 vector. The element drives expression of the eGFP reporter irrespective of whether it is introduced via AAV5 into animals at a neonatal (PND2) stage or during adulthood (8 weeks). FIG. 9B shows the quantified localization of eGFP expression, driven by the 1.7 kb element, and endogenous Glut1 protein, which labels the endothelial cells of the brain microvasculature. Greater than 80% of all Glut1 labeled brain capillaries in all three brain regions also expressed eGFP.

### DETAILED DESCRIPTION OF THE DISCLOSURE

The embodiments described in this disclosure can be combined in various ways. Any aspect or feature that is described for one embodiment can be incorporated into any other embodiment mentioned in this disclosure. While various novel features of the inventive principles have been shown, described and pointed out as applied to particular embodiments thereof, it should be understood that various omissions and substitutions and changes may be made by those skilled in the art without departing from the spirit of this disclosure. Those skilled in the art will appreciate that the inventive principles can be practiced in other than the described embodiments, which are presented for purposes of illustration and not limitation.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods and compositions of the present disclosure may be used to slow the development of disease symptoms or delay the onset of the disease or condition, or halt the progression of disease development. However, because not every treated subject may respond to a particular treatment protocol, regimen, process or remedy, treating does not require that the desired physiologic response or outcome be achieved in each and every subject or subject population, e.g., patient population. Accordingly, a given subject or subject population, e.g., patient population, may fail to respond or respond inadequately to treatment.

As used herein, the terms “ameliorate”, “ameliorating” and grammatical variations thereof mean to decrease the severity of the symptoms of a disease in a subject.

In the present disclosure, an “effective amount” of a therapeutic is an amount of such therapeutic that is sufficient to effect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of the subject, and like factors well known in the arts of, e.g., medicine and veterinary medicine. In general, a suitable dose of a therapeutic according to the disclosure will be that amount of the agent, which is the lowest dose effective to produce the desired effect with no or minimal side effects. The effective dose of a therapeutic according to the present disclosure may be administered as a single dose, or two, three, four, five, six or more sub-doses, administered separately at appropriate intervals throughout the treatment course (e.g., not necessarily on the same day).

The terminology used herein is for the purpose of describing particular embodiments only and is not intended to be limiting. As used in the specification and the appended claims, the singular forms “a,” “an,” and “the” include plural referents unless the context clearly dictates otherwise.

According to some aspects, the present disclosure provides a pharmaceutical composition comprising a recombinant adeno-associated virus (rAAV) vector carrying an agent that is effective to treat a brain endothelial dysfunction.

In some embodiments, the rAAV vector is a rAAV serotype 5 (AAV5) vector, a rAAV serotype rh74 (AAVrh74) vector, or a functional variant thereof. In some embodiments, a functional variant of the rAAV vector shares at least 90%, at least 91%, at least 92%, at least 93%, at least 94%, at least 95%, at least 96%, at least 97%, at least 98%, at least 99%, or 100% identity to any one of AAV5 and AAVrh74.

As used herein, the term “variant” or “functional variant” refer, interchangeably, to a protein (or a polynucleotide encoding such protein) that has one or more amino-acid substitutions, insertions, or deletion compared to a parental protein that retains one or more desired activities of the parental protein.

In some embodiments, the agent is selected from the group consisting of a small molecule, a biologic, a protein, an antisense RNA, a small interfering RNA (siRNA), a short hairpin RNA (shRNA), a polynucleotide sequence, or combinations thereof.

As one example, in some embodiments, the agent is an isolated nucleic acid comprising SLC2A1-NAT having a sequence selected from SEQ ID NO: 1 or SEQ ID NO: 2, or variants or fragments thereof. In the context of the present disclosure, variants and fragments of SEQ ID NOs: 1 and 2 include those having at least 50%, at least 60%, at least 70%, at least 75%, at least 80%, at least 85%, at least 90%, at least 95%, and at least 99% identity to any of SEQ ID NOs: 1 and 2.

As another example, in some embodiments, the agent is an isolated nucleic acid sequence encoding Glut1 or a functional variant thereof, operatively linked to a promoter. In some embodiments, the Glut1 comprises a sequence selected from SEQ ID NO: 3 or SEQ ID NO: 4. In some embodiments, the isolated nucleic acid sequence encoding Glut1 shares at least 75%, at least 80%, at least 85%, at least 90%, at least 95%, and at least 99% identity to any of SEQ ID NOs: 3 and 4. In some embodiments, the promoter is a Glut1 promoter or a chicken Beta-actin promoter. In some embodiments, the rAAV vector comprises a cytomegalovirus (CMV) enhancer.

The promoter as used in the present disclosure can also be any one of those described in US20230272422, the content of which is incorporated herein by reference. For example, in some embodiments, the promoter is an endothelial promoter that is not a Glut1 promoter, and it can be selected from the group consisting of a Tie-1 promoter, Tie-2 (TEK) promoter, FLT-1 promoter, FLK-1 (KDR) promoter, ICAM-2 promoter, VE-Cadherin (CDH5) promoter, VWF promoter, ENG promoter, PDGFB promoter, ESM1 promoter, APLN promoter, or Claudin-5 (Ple261) promoter.

In one example, in some embodiments, the promoter is a FLT-1 promoter. In some embodiments, the FLT-1 promoter is a human FLT-1 (hFLT-1) promoter. In some embodiments, the hFLT-1 promoter shares at least 75%, 80%, 85%, 90%, 95%, 96%, 97%, 98%, 99% or 100% identity with SEQ ID NO: 5.

In another example, in some embodiments, the promoter is a Tie-1 promoter. In some embodiments, the Tie-1 promoter is a human Tie-1 (hTie-1) promoter. In some embodiments, the hTie-1 promoter shares at least 75%, 80%, 85%, 90%, 95%, 96%, 97%, 98%, 99% or 100% identity with SEQ ID NO: 6.

In a further example, in some embodiments, the promoter is a vascular endothelial-cadherin (VE-cadherin) promoter. In some embodiments, the VE-cadherin promoter is a human VE-cadherin (hVE-cadherin) promoter. In some embodiments, the hVE-cadherin promoter shares at least 75%, 80%, 85%, 90%, 95%, 96%, 97%, 98%, 99% or 100% identity with SEQ ID NO: 7.

In some embodiments, the promoter is a ubiquitous promoter. In some embodiments, the promoter is a CMV promoter. In some embodiments, wherein the promoter is a CAG promoter.

In some embodiments of the present disclosure, the isolated nucleic acid sequence encoding Glut1 or a functional variant thereof comprises SEQ ID NO: 8 or SEQ ID NO: 9. In some embodiments, the promoter is a human Glut1 promoter element having the sequence of SEQ ID NO: 10. In some embodiments, the isolated nucleic acid sequence is fused with the promotor, having a sequence of SEQ ID NO: 11 or SEQ ID NO: 12.

As used herein, the term “isolated nucleic acid molecule” means a DNA or RNA of genomic, mRNA, cDNA, or synthetic origin or some combination thereof which is not associated with all or a portion of a polynucleotide in which the isolated polynucleotide is found in nature or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof or may include operably linked regulatory sequences that control expression of the coding region of the recited nucleic acid sequences, and/or may include vector sequences.

As used herein, the term “vector” includes any genetic element, such as a plasmid, phage, transposon, cosmid, chromosome, artificial chromosome, virus, or virion, which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. The phrase “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers. A “promoter” refers to a DNA sequence recognized by the synthetic machinery of the cell, or introduced synthetic machinery, required to initiate the specific transcription of a gene.

As used herein, the phrases “operatively positioned,” “operatively linked,” “under control,” or “under transcriptional control” means that the promoter is in the correct location and orientation in relation to the nucleic acid to control RNA polymerase initiation and expression of the gene.

As defined above, in the context of the present disclosure, Glucose transporter 1 (Glut1), also known as solute carrier family 2, facilitated glucose transporter member 1 (SLC2A1), is a uniporter protein that in humans is encoded by the SLC2A1 gene. Glut1 facilitates the transport of glucose across the plasma membranes of mammalian cells. “SLC2A1-NAT” refers to the natural antisense transcript that regulates the expression of SLCA1 or Glut1 gene, and it has two isoforms SLC2A1-NAT1 and SLC2A1-NAT2. SLC2A1-NAT1 (NAT1) and SLC2A1-NAT2 (NAT2) share exons 1 and 2, but whereas exon 3 in NAT1 is large (approximately 2.5 kb), its counterpart in NAT2 is much smaller (215 bp) and followed by a fourth exon of similar size. Thus, the cDNAs of the two transcripts are approximately 3.1 kb (NAT1) and approximately 1.1 kb (NAT2), and the corresponding genomic regions approximately 14 kb and 23 kb respectively. Accordingly, the present disclosure also provides for this natural antisense transcript denoted SLC2A1-NAT nucleic acid. In some embodiments, the nucleic acid is RNA. In some embodiments, the nucleic acid is DNA. In some embodiments, the nucleic acid is cDNA. In some embodiments, the SLC2A1-NAT is SLC2A1-NAT2. In some embodiments, SLC2A1-NAT comprises the nucleotide sequence SEQ ID NO: 1.

The present disclosure also provides for a nucleic acid comprising exons 1-3 of SLC2A1-NAT comprising the sequence SEQ ID NO: 2. In some embodiments, the nucleic acid is RNA. In some embodiments, the nucleic acid is DNA. In some embodiments, the nucleic acid is cDNA. As shown in US20210222167, the content of which is incorporated herein by reference, the exons 1-3 are sufficient to regulate expression of the SLC2A1 gene. For reference, exon 1 comprises nucleotides 1-529 of SEQ ID NOs: 1 and 2; exon 2 comprises nucleotides 530-675 of SEQ ID NOs: 1 and 2; and exon 3 comprises nucleotides 676-890 of SEQ ID NOs: 1 and 2.

The present disclosure also includes variants and fragments of the SLC2A1-NAT nucleic acids, including variants of SEQ ID NOs: 1 and 2, having at least 50%, at least 60%, at least 70%, at least 75%, at least 80%, at least 85%, at least 90%, at least 95%, and at least 99% identity to any of SEQ ID NOs: 1 and 2.

As used herein, the term “brain endothelial dysfunction” refers to a condition leads to, for example, oxidative stress, inflammation, increased vascular tone, blood-brain barrier (BBB) damage, and further thrombovascular complications in the brain. In some embodiments, the brain endothelial dysfunction is selected from the group consisting of a lysosomal storage disease, a neurodegenerative disease, and Glucose transporter 1 deficiency syndrome (GLUT1DS).

As used herein, non-limiting examples of a lysosomal storage disease include GM2 gangliosidosis, Niemann-Pick disease, Gaucher disease, Fabry disease, Metachromatic leukodystrophy, Globoid leukodystrophy, GM1 gangliosidosis, Multiple sulfatase deficiency, Alfa mannosidosis, Schindler disease, Aspartylglucosaminuria, Fucosidosis, Hurler syndrome, Scheie syndrome, Hurler-Scheie syndrome, Hunter syndrome, SanFilippo syndrome, Morquio syndrome, Maroteaux-Lamy syndrome, Sly syndrome, Galactosialidosis, Infantile sialic acid storage disease, Salla disease, Sialuria, Pseudo-Hurler-Polydystrophy, Pompe disease, Danon disease, and Cystinosis.

As used herein, non-limiting examples of a neurodegenerative disease include Alzheimer's disease, Parkinson's disease, Amyotrophic lateral sclerosis, Friedreich's ataxia, Multiple sclerosis, Huntington's Disease, Transmissible spongiform encephalopathy, Charcot-Marie-Tooth disease, Dementia with Lewy bodies, Corticobasal degeneration, Progressive supranuclear palsy, Chronic Traumatic Encephalopathy (CTE), and Hereditary spastic paraparesis.

According to some aspects, the present disclosure provides a method of treating or ameliorating the effect of a brain endothelial dysfunction in a subject, comprising administering to the subject an effective amount of any of the pharmaceutical compositions disclosed herein.

In some embodiments, the brain endothelial dysfunction is selected from the group consisting of a lysosomal storage disease, a neurodegenerative disease, and Glucose transporter 1 deficiency syndrome (GLUT1DS).

In some embodiments, the lysosomal storage disease is selected from the group consisting of GM2 gangliosidosis, Niemann-Pick disease, Gaucher disease, Fabry disease, Metachromatic leukodystrophy, Globoid leukodystrophy, GM1 gangliosidosis, Multiple sulfatase deficiency, Alfa mannosidosis, Schindler disease, Aspartylglucosaminuria, Fucosidosis, Hurler syndrome, Scheie syndrome, Hurler-Scheie syndrome, Hunter syndrome, SanFilippo syndrome, Morquio syndrome, Maroteaux-Lamy syndrome, Sly syndrome, Galactosialidosis, Infantile sialic acid storage disease, Salla disease, Sialuria, Pseudo-Hurler-Polydystrophy, Pompe disease, Danon disease, and Cystinosis.

In some embodiments, the neurodegenerative disease is selected from the group consisting of Alzheimer's disease, Parkinson's disease, Amyotrophic lateral sclerosis, Friedreich's ataxia, Multiple sclerosis, Huntington's Disease, Transmissible spongiform encephalopathy, Charcot-Marie-Tooth disease, Dementia with Lewy bodies, Corticobasal degeneration, Progressive supranuclear palsy, Chronic Traumatic Encephalopathy (CTE), and Hereditary spastic paraparesis.

As defined above, in the context of the present disclosure, a “subject” is a mammal, preferably, a human. In addition to humans, categories of mammals within the scope of the present disclosure include, for example, agricultural animals, veterinary animals, laboratory animals, etc. Some examples of agricultural animals include cows, pigs, horses, goats, etc. Some examples of veterinary animals include dogs, cats, etc. Some examples of laboratory animals include primates, rats, mice, rabbits, guinea pigs, etc. In some embodiments, the mammal is a non-rodent mammal. In some embodiments, the subject is a human. In some embodiments of the present disclosure, the phrase “a subject” means a subject having a brain endothelial dysfunction such as, e.g., Glucose transporter 1 deficiency syndrome (GLUT1DS).

In some embodiments, the subject has an intact blood-brain barrier. In some embodiments, the subject has an intact blood-brain barrier and the rAAV vector crosses the blood-brain barrier in sufficient amount to deliver the agent in endothelial cells lining the brain microvasculature.

In some embodiments, the pharmaceutical composition is administered by intracerebroventricular (ICV) injection. In some embodiments, the pharmaceutical composition is administered systemically. In some embodiments, the pharmaceutical composition is administered intravenously.

According to some aspects, the present disclosure provides a pharmaceutical composition comprising a recombinant adeno-associated virus serotype 5 (AAV5) vector carrying an isolated nucleic acid comprising SLC2A1-NAT having a sequence selected from SEQ ID NO: 1 or SEQ ID NO: 2, or variants or fragments thereof.

According to some aspects, the present disclosure provides a pharmaceutical composition comprising a recombinant adeno-associated virus serotype rh74 (AAVrh74) vector carrying an isolated nucleic acid comprising SLC2A1-NAT having a sequence selected from SEQ ID NO: 1 or SEQ ID NO: 2, or variants or fragments thereof.

According to some aspects, the present disclosure provides a pharmaceutical composition comprising a recombinant adeno-associated virus serotype 5 (AAV5) vector carrying an isolated nucleic acid sequence encoding Glut1 operatively linked to a promoter.

According to some aspects, the present disclosure provides a pharmaceutical composition comprising a recombinant adeno-associated virus serotype rh74 (AAVrh74) vector carrying an isolated nucleic acid sequence encoding Glut1 operatively linked to a promoter.

In some embodiments, the Glut1 comprises a sequence selected from SEQ ID NO: 3 or SEQ ID NO: 4. In some embodiments, the promoter is a Glut1 promoter or a chicken Beta-actin promoter. In some embodiments, the rAAV vector comprises a cytomegalovirus (CMV) enhancer.

In some embodiments, the isolated nucleic acid sequence encoding Glut1 or a functional variant thereof comprises SEQ ID NO: 8 or SEQ ID NO: 9. In some embodiments, the promoter is a human Glut1 promoter element having the sequence of SEQ ID NO: 10. In some embodiments, the isolated nucleic acid sequence is fused with the promotor, having a sequence of SEQ ID NO: 11 or SEQ ID NO: 12.

According to some aspects, the present disclosure provides a method of treating or ameliorating the effect of Glucose transporter 1 deficiency syndrome (GLUT1DS) in a subject, comprising administering to the subject an effective amount of any of the pharmaceutical composition disclosed herein.

In some embodiments, the subject is a mammal. In some embodiments, the mammal is selected from the group consisting of humans, veterinary animals, and agricultural animals. In some embodiments, the mammal is a non-rodent mammal. In some embodiments, the subject is a human.

In some embodiments, the subject has an intact blood-brain barrier. In some embodiments, the subject has an intact blood-brain barrier and the rAAV vector crosses the blood-brain barrier in sufficient amount to deliver the agent in endothelial cells lining the brain microvasculature.

In some embodiments, the pharmaceutical composition is administered by intracerebroventricular (ICV) injection. In some embodiments, the pharmaceutical composition is administered systemically. In some embodiments, the pharmaceutical composition is administered intravenously.

The following examples are provided to further illustrate the compositions and methods of the present disclosure. These examples are illustrative only and are not intended to limit the scope of the disclosure in any way.

### EXAMPLES

Methods for obtaining recombinant AAVs having a desired capsid protein have been described (See, for example, US 2003/0138772, the contents of which are incorporated herein by reference in their entirety). A number of different AAV capsid proteins have been described, for example, those disclosed in G. Gao, et al., J. Virol, 78(12):6381-6388 (June 2004); G. Gao, et al, Proc Natl Acad Sci USA, 100(10):6081-6086 (May 13, 2003); US 2003-0138772, US 2007/0036760, US 2009/0197338 the contents of which relating to AAVs capsid proteins and associated nucleotide and amino acid sequences are incorporated herein by reference. For examples disclosed herein, the AAV5 and AAVrh74 vectors and capsids are preferred.

### Example 1

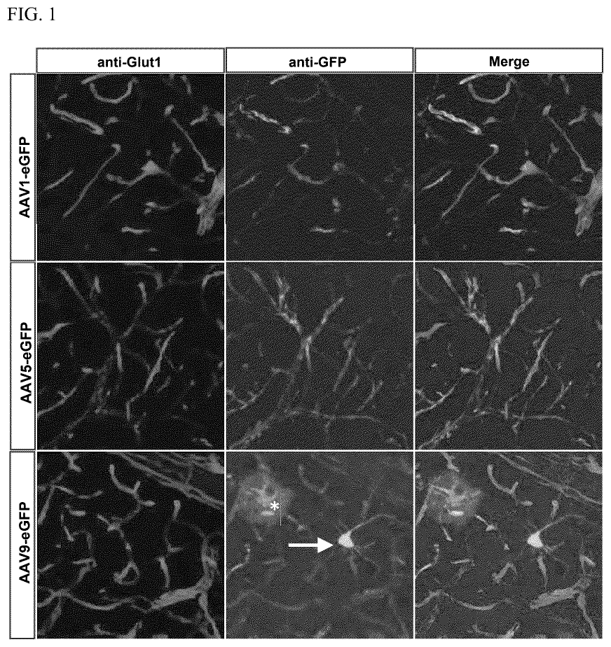

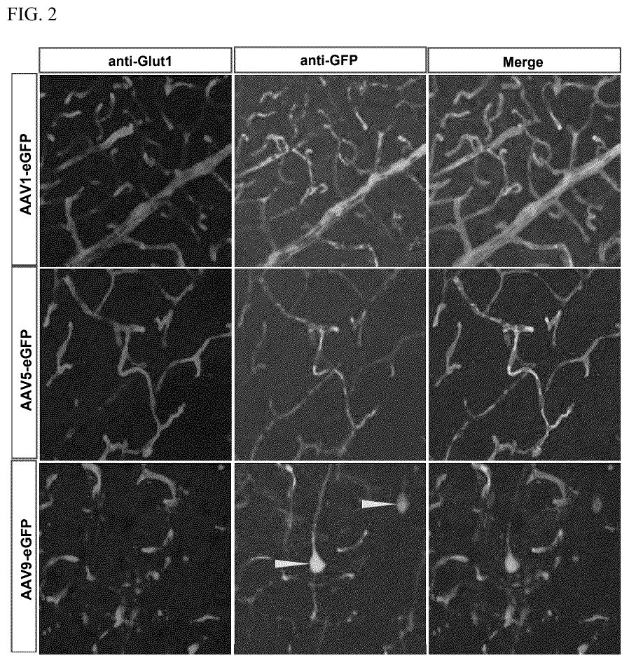

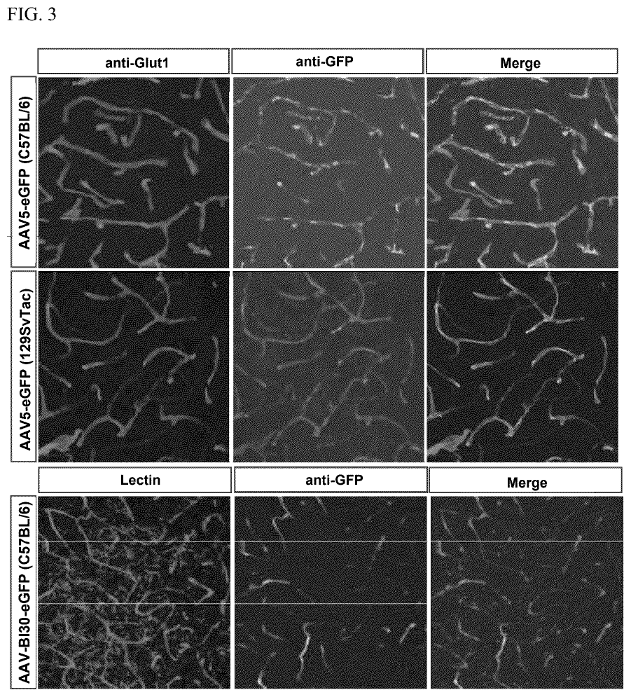

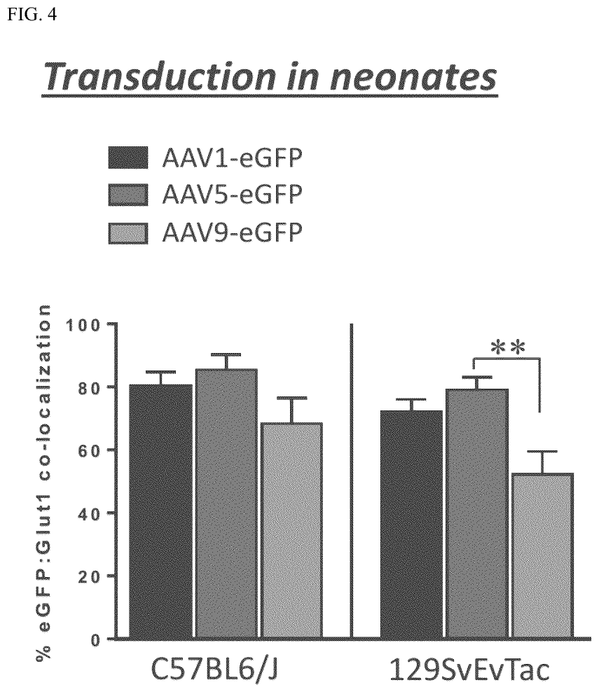

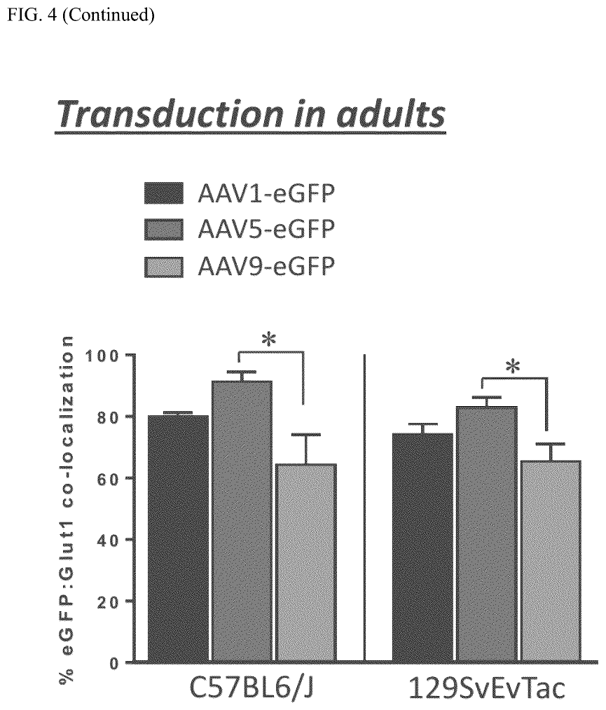

Results of experiments in which AAV1, AAV5, AAV9, AAVrh74 and AAV-BI30 are provided. AAVs 1, 5 and 9 were tested and compared for their ability to target brain endothelia in two strains of mice (C57BL/6 and 129SvEvTac) during neonatal and adult stages of life. As previously reported (PMID: 28106060, which is incorporated by reference as though recited in full herein), AAV9 targeted brain endothelia but also neurons and glia in the neonatal mice (FIGS. 1 & 2). In contrast, AAV5 was more selective and found to specifically target the brain microvasculature while de-targeting cells of the neuropil. This selectivity was maintained in both strains of mice during adulthood (FIG. 3). AAV-BI30, an engineered capsid, served as a control and efficiently targeted the brain microvasculature (FIG. 3) as previously reported (PMID: 35571675, which is incorporated by reference as though recited in full herein). Quantification of the relative efficiencies with which AAVs 1, 5 and 9 target the brain microvasculature indicate that AAV5 is the most efficient in each mouse strain and during neonatal and adult stages of life (FIG. 4).

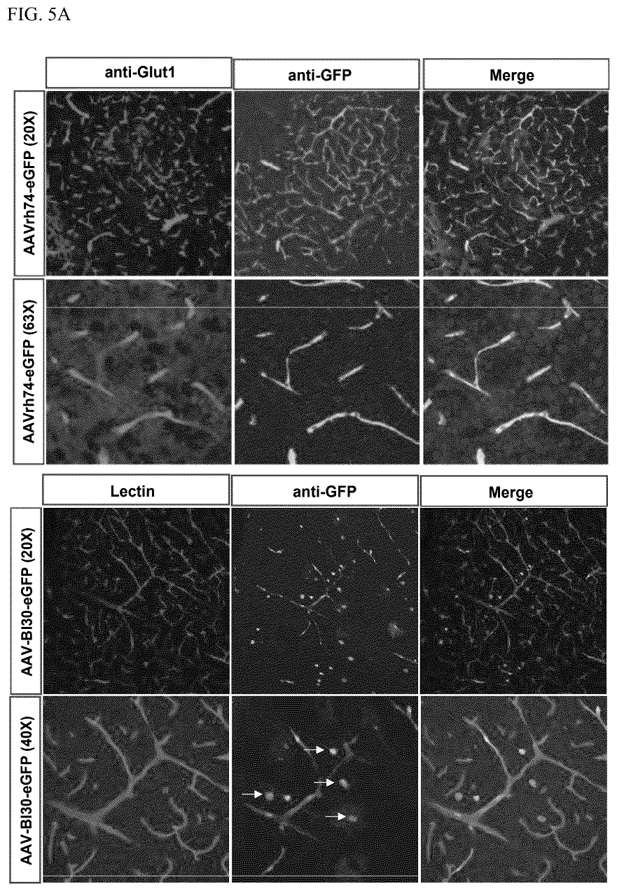

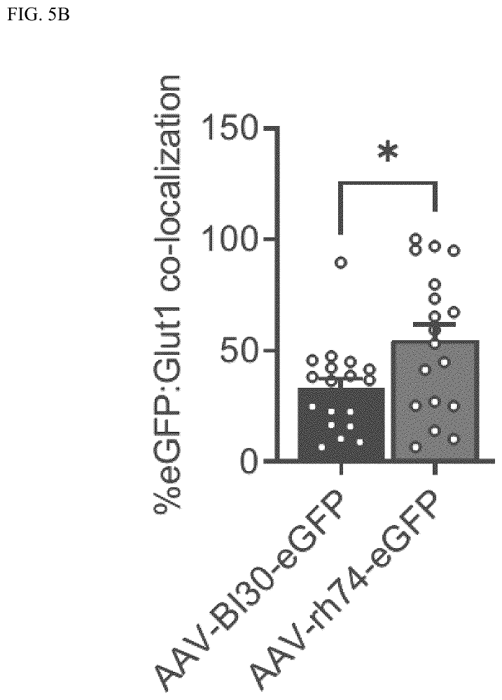

To examine the ability of AAVrh74 to target the brain endothelial cells, an AAVrh74-eGFP construct was systemically delivered to PND2 mice. An AAV-BI30-eGFP construct was tested in parallel for endothelial cell-specific tropism. Whereas AAV-BI30 was found to target brain neurons and the cerebral endothelial cells, AAVrh74 was more selective and mostly targeted the brain microvasculature (FIG. 5A). Moreover, a comparison the relative efficiencies of endothelial cell targeting by the two AAV serotypes indicated that AAVrh74 is significantly more efficient than AAVBI30 (FIG. 5B). These data raise the potential for the use of AAV5 and AAVrh74 for targeting human brain endothelial cells for the treatment of neurovascular and neurodegenerative conditions.

### Example 2

AAVrh74 and AAV4 vectors can be engineered to carry a therapeutic agent that are known and tested for efficacy, such as, e.g., the nucleic acid comprising SLC2A1-NAT as described in US20210222167, or the nucleic acid sequence encoding Glut1 operatively linked to a promoter as described in US20210069292 and US20230272422, the contents of which are incorporated herein by reference in their entirety. The rAAV vectors disclosed herein can also be engineered to carry any therapeutic agent that is under development for the treatment of a brain endothelial dysfunction such as a lysosomal storage disease, a neurodegenerative disease, and Glucose transporter 1 deficiency syndrome (GLUT1DS).

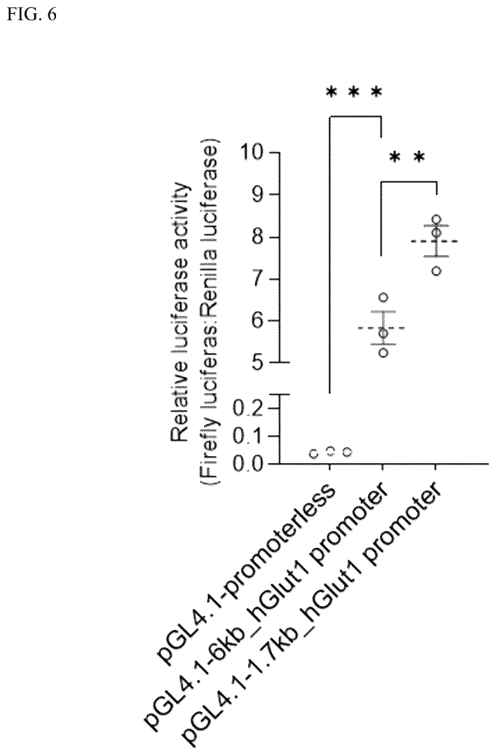

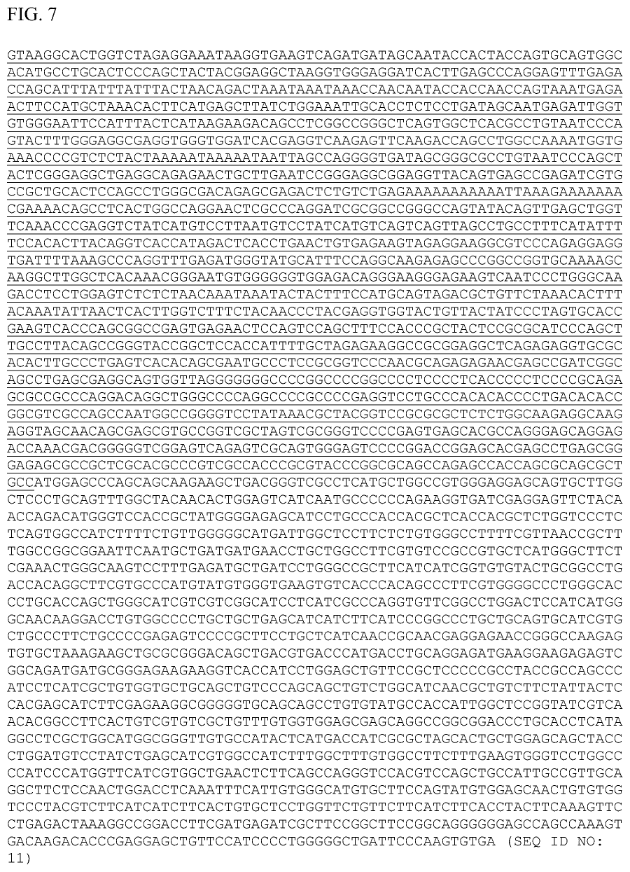

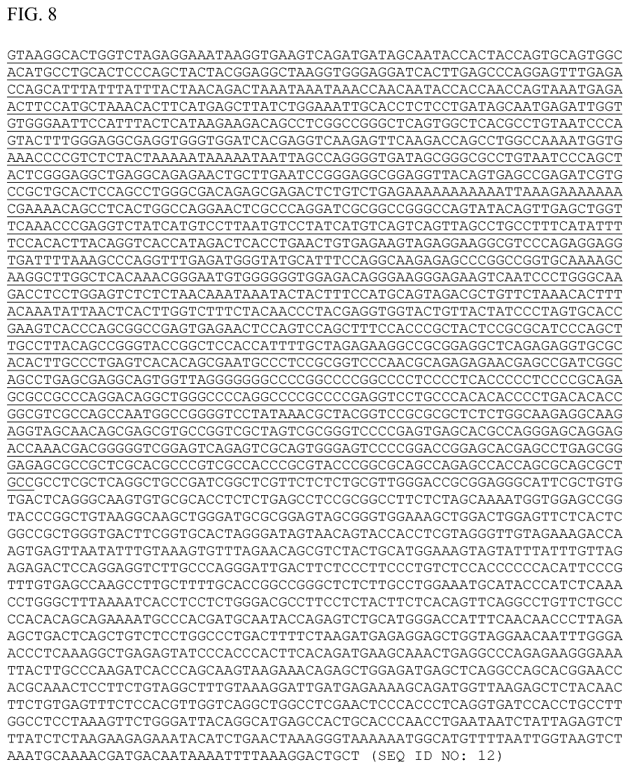

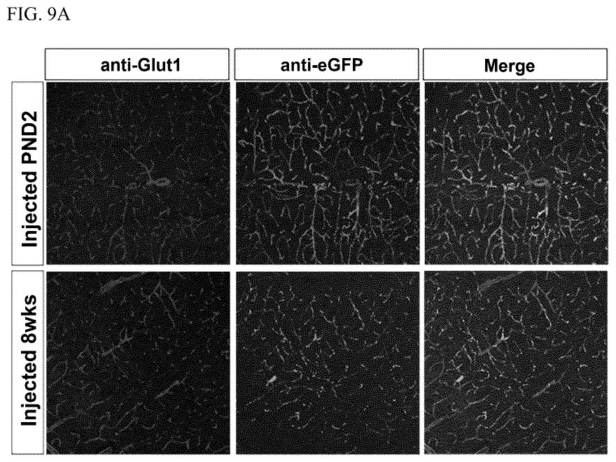

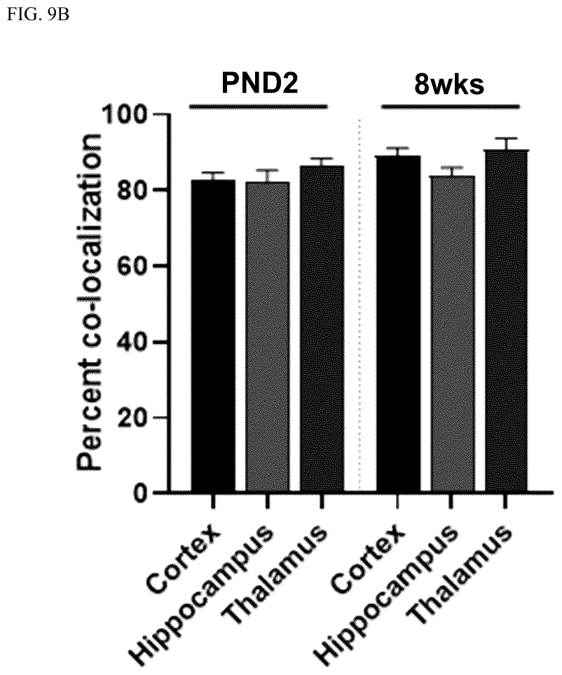

A new Glut1 promoter was developed and tested in a rAAV vector for the treatment of Glut1DS and other conditions characterized by low Glut1 (FIG. 6). This promoter is a 1.7 kb human Glut1 promoter element, which drives robust expression of a reporter in vivo in brain endothelial cells (FIGS. 9A and 9B). As shown in FIG. 9A, after injected to animals, the promoter element drove significant expression of an eGFP reporter in an AAV5 vector, and such promoted expression is irrespective of whether this promoter element was introduced via AAV5 into animals at a neonatal (PND2) stage or during adulthood (8 weeks). By quantifying the localization of eGFP expression, driven by the 1.7 kb element, and endogenous Glut1 protein, which labeled the endothelial cells of the brain microvasculature, it was found that greater than 80% of all Glut1 labeled brain capillaries in all three brain regions also expressed eGFP. These results demonstrated that this new 1.7 kb human Glut1 promoter element, when fused to a target gene (e.g., human Glut1 gene (FIG. 7), or human SLC2A1-DT natural antisense transcript (FIG. 8)), would raise Glut1 and thereby treat a brain endothelial dysfunction such as Glucose transporter 1 deficiency syndrome (GLUT1DS).

All documents cited in this application are hereby incorporated by reference as if recited in full herein. In the event of a conflict between the teachings of this application and those of the incorporated documents, the teachings of this application control.

Although illustrative embodiments of the present disclosure have been described herein, it should be understood that the disclosure is not limited to those described, and that various other changes or modifications may be made by one skilled in the art without departing from the scope or spirit of the disclosure.

1. A pharmaceutical composition comprising a recombinant adeno-associated virus (rAAV) vector carrying an agent that is effective to treat a brain endothelial dysfunction.

2. The pharmaceutical composition of claim 1, wherein the rAAV vector is a rAAV serotype 5 (AAV5) vector, a rAAV serotype rh74 (AAVrh74) vector, or a functional variant thereof.

3. The pharmaceutical composition of claim 1, wherein the agent is selected from the group consisting of a small molecule, a biologic, a protein, an antisense RNA, a small interfering RNA (siRNA), a short hairpin RNA (shRNA), a polynucleotide sequence, or combinations thereof.

4. The pharmaceutical composition of claim 1, wherein the agent is an isolated nucleic acid comprising SLC2A1-NAT having a sequence selected from SEQ ID NO: 1 or SEQ ID NO: 2, or variants or fragments thereof.

5. The pharmaceutical composition of claim 1, wherein the agent is an isolated nucleic acid sequence encoding Glut1 or a functional variant thereof, operatively linked to a promoter.

6. The pharmaceutical composition of claim 5, wherein the Glut1 comprises a sequence selected from SEQ ID NO: 3 or SEQ ID NO: 4.

7. The pharmaceutical composition of claim 5, wherein the promoter is a Glut1 promoter or a chicken Beta-actin promoter.

8. The pharmaceutical composition of claim 1, wherein the rAAV vector comprises a cytomegalovirus (CMV) enhancer.

9. The pharmaceutical composition of claim 5, wherein the promoter is an endothelial promoter selected from the group consisting of a Tie-1 promoter, Tie-2 (TEK) promoter, FLT-1 promoter, FLK-1 (KDR) promoter, ICAM-2 promoter, VE-Cadherin (CDH5) promoter, VWF promoter, ENG promoter, PDGFB promoter, ESM1 promoter, APLN promoter, or Claudin-5 (Ple261) promoter, provided the endothelial promoter is not a Glut1 promoter.

10. The pharmaceutical composition of claim 5, wherein the promoter is a FLT-1 promoter.

11. The pharmaceutical composition of claim 10, wherein the FLT-1 promoter is a human FLT-1 (hFLT-1) promoter.

12. The pharmaceutical composition of claim 11, wherein the hFLT-1 promoter shares at least 75%, 80%, 85%, 90%, 95%, 96%, 97%, 98%, 99% or 100% identity with SEQ ID NO: 5.

13. The pharmaceutical composition of claim 5, wherein the promoter is a Tie-1 promoter.

14. The pharmaceutical composition of claim 13, wherein the Tie-1 promoter is a human Tie-1 (hTie-1) promoter.

15. The pharmaceutical composition of claim 14, wherein the hTie-1 promoter shares at least 75%, 80%, 85%, 90%, 95%, 96%, 97%, 98%, 99% or 100% identity with SEQ ID NO: 6.

16. The pharmaceutical composition of claim 5, wherein the promoter is a vascular endothelial-cadherin (VE-cadherin) promoter.

17. The pharmaceutical composition of claim 16, wherein the VE-cadherin promoter is a human VE-cadherin (hVE-cadherin) promoter.

18. The pharmaceutical composition of claim 17, wherein the hVE-cadherin promoter shares at least 75%, 80%, 85%, 90%, 95%, 96%, 97%, 98%, 99% or 100% identity with SEQ ID NO: 7.

19. The pharmaceutical composition of claim 5, wherein the promoter is a ubiquitous promoter.

20. The pharmaceutical composition of claim 5, wherein the promoter is a CMV promoter.

21. The pharmaceutical composition of claim 5, wherein the promoter is a CAG promoter.

22. The pharmaceutical composition of claim 5, wherein the isolated nucleic acid sequence comprises SEQ ID NO: 8 or SEQ ID NO: 9.

23. The pharmaceutical composition of claim 5, wherein the promoter is a human Glut1 promoter element having the sequence of SEQ ID NO: 10.

24. The pharmaceutical composition of claim 5, wherein the isolated nucleic acid sequence is fused with the promotor, having a sequence of SEQ ID NO: 11 or SEQ ID NO: 12.

25. The pharmaceutical composition of claim 1, wherein the brain endothelial dysfunction is selected from the group consisting of a lysosomal storage disease, a neurodegenerative disease, and Glucose transporter 1 deficiency syndrome (GLUT1DS).

26. The pharmaceutical composition of claim 25, wherein the lysosomal storage disease is selected from the group consisting of GM2 gangliosidosis, Niemann-Pick disease, Gaucher disease, Fabry disease, Metachromatic leukodystrophy, Globoid leukodystrophy, GM1 gangliosidosis, Multiple sulfatase deficiency, Alfa mannosidosis, Schindler disease, Aspartylglucosaminuria, Fucosidosis, Hurler syndrome, Scheie syndrome, Hurler-Scheie syndrome, Hunter syndrome, SanFilippo syndrome, Morquio syndrome, Maroteaux-Lamy syndrome, Sly syndrome, Galactosialidosis, Infantile sialic acid storage disease, Salla disease, Sialuria, Pseudo-Hurler-Polydystrophy, Pompe disease, Danon disease, and Cystinosis.

27. The pharmaceutical composition of claim 25, wherein the neurodegenerative disease is selected from the group consisting of Alzheimer's disease, Parkinson's disease, Amyotrophic lateral sclerosis, Friedreich's ataxia, Multiple sclerosis, Huntington's Disease, Transmissible spongiform encephalopathy, Charcot-Marie-Tooth disease, Dementia with Lewy bodies, Corticobasal degeneration, Progressive supranuclear palsy, Chronic Traumatic Encephalopathy (CTE), and Hereditary spastic paraparesis.

28. A method of treating or ameliorating the effect of a brain endothelial dysfunction in a subject, comprising administering to the subject an effective amount of the pharmaceutical composition according to claim 1.

29. The method of claim 28, wherein the brain endothelial dysfunction is selected from the group consisting of a lysosomal storage disease, a neurodegenerative disease, and Glucose transporter 1 deficiency syndrome (GLUT1DS).

30. The method of claim 29, wherein the lysosomal storage disease is selected from the group consisting of GM2 gangliosidosis, Niemann-Pick disease, Gaucher disease, Fabry disease, Metachromatic leukodystrophy, Globoid leukodystrophy, GM1 gangliosidosis, Multiple sulfatase deficiency, Alfa mannosidosis, Schindler disease, Aspartylglucosaminuria, Fucosidosis, Hurler syndrome, Scheie syndrome, Hurler-Scheie syndrome, Hunter syndrome, SanFilippo syndrome, Morquio syndrome, Maroteaux-Lamy syndrome, Sly syndrome, Galactosialidosis, Infantile sialic acid storage disease, Salla disease, Sialuria, Pseudo-Hurler-Polydystrophy, Pompe disease, Danon disease, and Cystinosis.

31. The method of claim 29, wherein the neurodegenerative disease is selected from the group consisting of Alzheimer's disease, Parkinson's disease, Amyotrophic lateral sclerosis, Friedreich's ataxia, Multiple sclerosis, Huntington's Disease, Transmissible spongiform encephalopathy, Charcot-Marie-Tooth disease, Dementia with Lewy bodies, Corticobasal degeneration, Progressive supranuclear palsy, Chronic Traumatic Encephalopathy (CTE), and Hereditary spastic paraparesis.

32. The method of claim 28, wherein the subject is a mammal.

33. The method of claim 32, wherein the mammal is selected from the group consisting of humans, veterinary animals, and agricultural animals.

34. The method of claim 32, wherein the mammal is a non-rodent mammal.

35. The method of claim 28, wherein the subject is a human.

36. The method of claim 28, wherein the subject has an intact blood-brain barrier.

37. The method of claim 28, wherein the subject has an intact blood-brain barrier and the rAAV vector crosses the blood-brain barrier in sufficient amount to deliver the agent in endothelial cells lining the brain microvasculature.

38. The method of claim 28, wherein the pharmaceutical composition is administered by intracerebroventricular (ICV) injection.

39. The method of claim 28, wherein the pharmaceutical composition is administered systemically.

40. The method of claim 28, wherein the pharmaceutical composition is administered intravenously.

41. A pharmaceutical composition comprising a recombinant adeno-associated virus serotype 5 (AAV5) vector carrying an isolated nucleic acid comprising SLC2A1-NAT having a sequence selected from SEQ ID NO: 1 or SEQ ID NO: 2, or variants or fragments thereof.

42. A pharmaceutical composition comprising a recombinant adeno-associated virus serotype rh74 (AAVrh74) vector carrying an isolated nucleic acid comprising SLC2A1-NAT having a sequence selected from SEQ ID NO: 1 or SEQ ID NO: 2, or variants or fragments thereof.

43. A pharmaceutical composition comprising a recombinant adeno-associated virus serotype 5 (AAV5) vector carrying an isolated nucleic acid sequence encoding Glut1 operatively linked to a promoter.

44. A pharmaceutical composition comprising a recombinant adeno-associated virus serotype rh74 (AAVrh74) vector carrying an isolated nucleic acid sequence encoding Glut1 operatively linked to a promoter.

45. The pharmaceutical composition of claim 43, wherein the Glut1 comprises a sequence selected from SEQ ID NO: 3 or SEQ ID NO: 4.

46. The pharmaceutical composition of claim 43, wherein the promoter is a Glut1 promoter or a chicken Beta-actin promoter.

47. The pharmaceutical composition of claim 43, wherein the rAAV vector comprises a cytomegalovirus (CMV) enhancer.

48. The pharmaceutical composition of claim 43, wherein the isolated nucleic acid sequence comprises SEQ ID NO: 8 or SEQ ID NO: 9.

49. The pharmaceutical composition of claim 48, wherein the promoter is a human Glut1 promoter having the sequence of SEQ ID NO: 10.

50. The pharmaceutical composition of claim 48, wherein the isolated nucleic acid sequence is fused with the promotor, having a sequence of SEQ ID NO: 11 or SEQ ID NO: 12.

51. A method of treating or ameliorating the effect of Glucose transporter 1 deficiency syndrome (GLUT1DS) in a subject, comprising administering to the subject an effective amount of the pharmaceutical composition according to claim 41.

52. The method of claim 51, wherein the subject is a mammal.

53. The method of claim 52, wherein the mammal is selected from the group consisting of humans, veterinary animals, and agricultural animals.

54. The method of claim 52, wherein the mammal is a non-rodent mammal.

55. The method of claim 51, wherein the subject is a human.

56. The method of claim 51, wherein the subject has an intact blood-brain barrier.

57. The method of claim 51, wherein the subject has an intact blood-brain barrier and the rAAV vector crosses the blood-brain barrier in sufficient amount to deliver the agent in endothelial cells lining the brain microvasculature.

58. The method of claim 51, wherein the pharmaceutical composition is administered by intracerebroventricular (ICV) injection.

59. The method of claim 51, wherein the pharmaceutical composition is administered systemically.

60. The method of claim 51, wherein the pharmaceutical composition is administered intravenously.
